(19) United States
(12) Patent Application Publication
(10) Pub. No.: US 2021/0279657 A1
(43) Pub. Date: Sep. 9, 2021

(54) SYSTEMS AND METHODS FOR MAINTAINING WORKSITE SAFETY

(71) Applicant: Christopher Golden, Ladera Ranch, CA (US)

(72) Inventor: Christopher Golden, Ladera Ranch, CA (US)

(21) Appl. No.: 17/178,191

(22) Filed: Feb. 17, 2021

Related U.S. Application Data
(60) Provisional application No. 62/977,629, filed on Feb. 17, 2020.

(51) Int. Cl. G06Q 10/06; G06Q 50/26; G16H 40/67; G16H 10/60; G06N 20/00; G01D 21/02; G08B 21/18

(52) U.S. Cl. CPC G06Q 10/0635; G06Q 10/06313; G06Q 10/0639; G06Q 50/26; G08B 21/18; G16H 10/60; G06N 20/00; G01D 21/02; G16H 40/67

(57) ABSTRACT

A system and method for maintaining worksite safety is provided. The system includes a plurality of sensors communicatively coupled to a server configured to collect environmental data within a worksite in order to monitor components of the worksite. The system further includes a machine learning server configured to utilize the environmental data in one or more machine learning algorithms in order to generate predictions associated with the components. Monitoring, efficiency scoring, and operational adjustments are performed by the server based on the predictions of the machine learning server.

### CROSS-REFERENCE TO RELATED APPLICATIONS

This application claims priority to U.S. Provisional Patent Application Ser. No. 62/977,629 filed Feb. 17, 2020, and claims the benefit of that application, the entirety of which is hereby incorporated by reference.

### FIELD OF THE INVENTION

The present invention relates generally to the field of collecting, monitoring, and evaluating workplace safety and productivity and automatically applying implementations to improve overall safety and productivity.

### BACKGROUND OF THE INVENTION

Historically, monitoring of workplaces and measuring workplace safety has been tasked to one or more individuals that serve at a supervisory and/or managerial level. Nonetheless, a common issue associated with this approach is the susceptibility to human error, which in certain circumstances can be catastrophic if not handled in an appropriate timeframe or detected altogether. For example, lack of detecting overheating equipment by the aforementioned individual could result in damage to the equipment and/or damage to personnel within the workplace.

Sensors, wearable technology, and other applicable mechanisms have recently become integrated in various environments in order to increase the accessibility and quality of data collected in a worksite. However, there have been issues associated with fully integrating the aforementioned mechanisms into worksites due to the inability to correlate the physical motions and actions of personnel on the worksite with operational conditions of components of the worksite such as tools, equipment, and utilities. In addition, there currently does not exist any mechanisms configured to account for the specific physical and biometric attributes of personnel in the workspace and whether these attributes contribute to or affect the efficiency of the worksite and the overall safety of the worksite.

Therefore, a need exists to overcome the problems with the prior art as discussed above. In particular, what is needed is a system and method to not only collect various forms of data within a worksite in a scalable manner, but also provide efficient monitoring and maintenance of the worksite and its overall safety.

### SUMMARY OF THE INVENTION

The invention provides a system and method for maintaining worksite safety that overcomes the hereinabove-mentioned disadvantages of the heretofore-known devices and methods of this general type and that effectively increases worksite performance and overall safety.

With the foregoing and other objects in view, there is provided, in accordance with the invention, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one implementation, the system for maintaining worksite safety includes a plurality of sensors, a computing device communicatively coupled to the plurality of sensors, wherein the computing device includes a non-transitory memory storing an executable code and a hardware processor executing the executable code to receive a plurality of environmental data associated with a worksite from one or more of the plurality of sensors, extract at least one identifier associated with a component of the worksite from the plurality of environmental data, identify an operational threshold based on the plurality of environmental data, determine, based on the identifier, whether the component exceeds the operational threshold, and transmit a notification based on the determination. Other implementations of this aspect include corresponding computer systems, apparatus, and computer programs recorded on one or more computer storage devices, each configured to perform the actions of the methods. In some implementations, the plurality of sensors include a plurality of nodes affixed to the applicable component of the worksite in order to acquire data associated with the component.

In other implementations, the system the computing device is associated with a plurality of nodes communicatively coupled via a network.

In other implementations, the plurality of environmental data comprises at least one of a temperature, an atmospheric humidity, an air quality measurement, an operational status, a windspeed, a biometric reading, a movement, a change of movement, an audio input, or a toxins reading.

In other implementations, the computing device is further configured to determine an efficiency score associated with the component based on the plurality of environmental data.

In other implementations, the computing device is further configured to generate a logistics schedule based on the efficiency score and the plurality of environmental data.

In other implementations, the plurality of sensors comprises at least one of a camera, a scanner, an accelerometer, a gyroscope, a microphone, a thermometer, a measurement device, a transducer, a pressure switch, a capacitance switch, an electrochemical detector, a semiconductor measurer, an air quality sensor, wind speed sensor, a pressure sensor, or a photo sensor.

In other implementations, the component is at least one of an individual, a safety equipment, a computing device, and a utility equipment, and wherein the component is associated with the worksite.

In other implementations, the computing device is further configured to store training data that comprises a plurality of training instances, wherein each of the plurality of training instances includes a plurality of feature values and a label that indicates whether the training instance pertains to the component, and use one or more machine learning techniques to train a classification model based on the training data.

In other implementations, the computing device is further configured to notify a first responder based on an alert indicating the identifier exceeding the operational threshold is a hazard associated with the worksite.

In one implementation, the method for maintaining worksite safety includes receiving a plurality of environmental data associated with a worksite from one or more of a plurality of sensors, identifying, using at least a computing device communicative coupled to the network, at least one identifier associated with a component of the worksite from the plurality of environmental data, identifying, using the computing device, an operational threshold associated with the component based on the plurality of environmental data, determining, using the computing device, whether the component exceeds the operational threshold based on the identifier, and transmitting, using the computing device, a notification based on the determination. Implementations of the described techniques may include hardware, a method or process, or computer software on a computer-accessible medium. In other implementations, the method may include determining whether the component exceeds the operational threshold based on the identifier and may further comprise applying, via the computing device, an adjustment action to the component via at least one node of the plurality of nodes.

In another implementation, the system for maintaining worksite safety includes a computer system comprising: a non-transitory memory storing an executable code, a vital measurement database storing a plurality of vital measurement data, and at least one of an environmental measurement database storing a plurality of environmental measurement data and a tool measurement database storing a plurality of tool measurement data, and a hardware processor executing the executable code to receive a first input from a first sensor, the first input being a vital measurement, compare the first input with the vital measurement data stored in the vital measurement database, determine that the first input is outside a standard range for the vital measurement based on comparison, receive a second input from a second sensor, the second input being one of an environmental measurement and a tool measurement, compare the second input with the corresponding environmental measurement data stored in the environmental measurement database or the corresponding tool measurement data stored in the tool measurement database, determine the second input is outside a standard range based on the comparison, identify a safety condition based on a coincidence of the first input and the second input each being outside a standard range, and transmit an alert based on the identified safety condition.

Although the invention is illustrated and described herein as embodied in a system and method for managing worksites, it is, nevertheless, not intended to be limited to the details shown because various modifications and structural changes may be made therein without departing from the spirit of the invention and within the scope and range of equivalents of the claims. Additionally, well-known elements of exemplary implementations of the invention will not be described in detail or will be omitted so as not to obscure the relevant details of the invention.

Other features that are considered as characteristic for the invention are set forth in the appended claims. As required, detailed implementations of the present invention are disclosed herein; however, in some implementations, the disclosed implementations are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, in some implementations, the terminology used herein is for the purpose of describing particular implementations only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

In the description of the implementations of the present invention, it should be noted that, unless otherwise clearly defined and limited, terms such as “installed”, “coupled”, “connected” should be broadly interpreted, for example, it may be fixedly connected, or may be detachably connected, or integrally connected; it may be mechanically connected, or may be electrically connected; it may be directly connected or may be indirectly connected via an intermediate medium. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system. Those skilled in the art can understand the specific meanings of the above-mentioned terms in the implementations of the present invention according to the specific circumstances.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various implementations and explain various principles and advantages all in accordance with the present invention.

FIG. 1 is a block diagram depicting an exemplary system for maintaining workplace safety, according to one implementation of the present disclosure;

FIG. 2 is a block diagram depicting various modules utilized by the system for maintaining workplace safety, according to one implementation of the present disclosure;

FIG. 3A is a diagram depicting an apparatus utilized by a system for maintaining workplace safety, according to an exemplary implementation;

FIG. 3B is another view of the apparatus shown in FIG. 3;

FIG. 4 is a block diagram illustrating an exemplary method for maintaining workplace safety, according to one implementation of the present disclosure;

FIG. 5 illustrates a computer system according to exemplary implementations of the present technology, according to one implementation of the present disclosure; and

FIG. 6 is a flowchart showing an exemplary method of for maintaining workplace safety, according to an exemplary implementation.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. In some implementations, the disclosed implementations are merely exemplary of the invention, which can be embodied in various forms.

The present invention provides a novel and efficient system and method for maintaining worksite safety configured to actively collect data from sensors and nodes from a worksite in real-time and aggregate the collected data in a scalable manner in order to generate operational thresholds associated with components of the workplace. These thresholds may be utilized to determine an efficiency rate of a component of the workplace associated with the applicable sensor or node or utilized in order to allow a processor communicatively coupled to the sensors and nodes to perform functions on components within the worksite in order to increase the overall safety of the worksite. Implementations of the invention provide wearable devices and affixable sensors configured to be attached to utilities, equipment, individuals, etc., associated with the worksite in order to continuously optimize the performance of a worksite and its components.

In some implementations data collected by the sensors and nodes may be entered into a machine learning algorithm in order to generate predictions associated with components of the worksite, wherein the predictions may be used by the processor to automatically generate alerts and notifications relating to issues, inefficiencies, and/or emergencies of the worksite, and subsequently automatically apply functions to rectify the issues and/or inefficiencies of the worksite. The system and methods described herein are configured to increase and optimize performance and safety within a workplace via the scalable collection of big data, optimizing of said data in real-time, and application of functions to components of the worksite based on predictions and analysis derived from the data in a scalable manner. Thus, by automated and scalable processing of the collected data in real-time, the processing cost over network, computation, and storage is reduced in a manner that simultaneously not only maximizes the performance of data processing, but also improves worksite safety and performance via application of the processed data to worksite components.

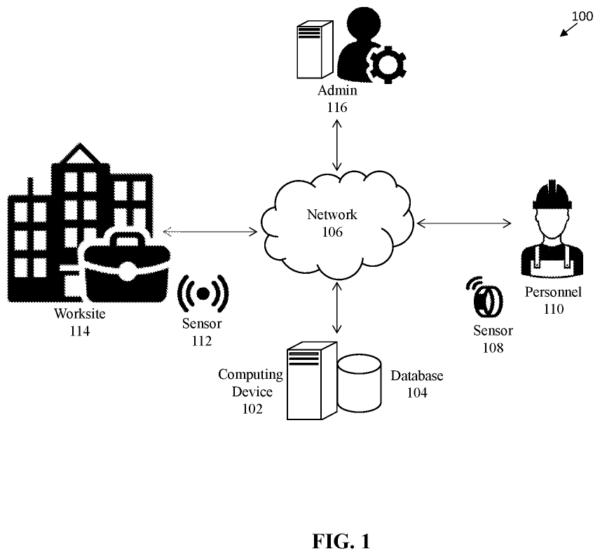

Referring now to FIG. 1, a system for maintaining worksite safety 100 is depicted, according to an exemplary implementation. In one implementation, system 100 includes a computing device 102 communicatively coupled to a database 104, a communicative network 106, a first sensor 108 configured to be affixed to a personnel 110, a second sensor 112 configured to be associated with a worksite 114, and an administrator 116 associated with worksite 114. In some implementations, computing device 102, first sensor 108, second sensor 112, and administrator 116 are communicatively coupled via network 106. Computing device 102 may be a server, a networked computer, a laptop computer, a tablet computer, a mobile phone, a smart device, such as a smartphone or a smartwatch, or a computing device included in a tool or construction equipment located at or within worksite 114. In some implementations, first sensor 108 and second sensor 112 may be one or more of a gyroscope, accelerometer, infrared sensor, proximity sensor, position sensor, biometric data sensor, pressure sensor, vision/imaging sensor, measurement device, microphone, transducer, capacitance switch, pressure switch, scanner, gas/chemical detector, temperature sensor, radiation sensor, photoelectric sensor, particle sensor, motion sensor, leak sensor, humidity sensor, air quality sensor, semiconductor measurer, air quality sensor, wind speed sensor or any other applicable sensor configured to collect data.

In one implementation, data collected and/or processed by any of the aforementioned is configured to be analyzed and/or presented on a centralized platform generated by computing device 102 allowing a user or administrator 116 to have access to data or analyses based on data collected by computing device 102, first sensor 108, and/or second sensor 112. In some implementations, the centralized platform provided by computing device 102 is configured to include various and/or tiered versions allowing users of the centralized platform to have varying access to particular data sets based upon the applicable entity. For example, administrator 116 is configured to have access to all data collected by first sensor 108 and second sensor 112 configured to contribute to or take away from the efficiency and overall safety of worksite 114. In some implementations, network 106 is configured to communicate with a network of computing devices including one or more nodes (network nodes) configured to communicate with or function as first sensor 108 and second sensor 112, wherein the nodes may each transmit and receive signals using wireless technology such as Wi-Fi, Bluetooth, Bluetooth Low Energy (BLE), long range radio frequency (LoRa) technology, radio frequency identification (RFID) active and passive RDID tags, mobile phone connectivity, such as cellular, satellite communications, LTE, etc.

In some implementations, the one or more nodes include a computer processor, computer memory, and wireless connectivity technology, such as LTE, 3G, 2.4 GHZ & 5.0 GHz, Mesh, BLE, BLE Mesh, LoRaWAN, GPS, etc. The computer processor may be a hardware processor. The computer memory may be a non-transitory memory. In some implementations the one or more nodes may be computing devices, wherein examples of computing devices include a laptop computer, a tablet computer, a smartphone, a desktop computer, a Personal Digital Assistant (PDA), and any other mechanism including a hardware processor configured to support sending and receiving wireless communication signals. System 100 illustrates only one of many possible arrangements of components configured to perform the functionality described herein. Other arrangements may include fewer or different components, and the division of work between the components may vary depending on the arrangement.

Computing device 102 may be implemented in hardware, software, or a combination of hardware and software. Network 106 may be a wireless local area network (WLAN), wireless personal area network (WPAN), wireless wide area network (WWAN), universal mobile telecommunications service (UMTS), enhanced packet system (EPS), new radio wireless network (NR), internet, LTE, GSM, WCDMA, 3rd generation partnership project (3GPP), a combination of more than one network and/or more than one type of network, or any other applicable communications network.

In some implementations, first sensor 108 and second sensor 112 (and/or one or more nodes) are configured to be affixed and/or associated with one or more components of worksite 114, wherein first sensor 108 and second sensor 112 may be configured to be included in an article of clothing, protective gear, googles, hardhat, gloves, securing strap/band/belt, or any other applicable apparel and may be configured to be wearable allowing collected data to be transmitted to computing device 102 over network 106. In some implementations, the one or more nodes are distributed throughout worksite 114 to provide complete wireless coverage such that personnel 110 will be within wireless communication range of at least one wireless-enabled network node whenever personnel 110 is within a boundary of worksite 114. When personnel 110 is located within and/or proximate to worksite 114, first sensor 108 is continuously collecting data associated with personnel 110 and transmitting the collected data to computing device 102 over network 106. For example, first sensor 108 may be collecting motion data and/or haptic data associated with personnel 110 while within worksite 114 while simultaneously tracking the actions/movements of personnel 110 relative to second sensor 112, and continuously transmitting the collected data to computing device 102 over network 106.

In some implementations, in some implementations, first sensor 108 and second sensor 112 may be configured to be allocated and associated with a component of worksite 114 allowing the respective collected data to be transmitted to computing device 102 in order to determine analytics for worksite 114. As described herein, a component of worksite 114 includes, but is not limited to personnel, goods/merchandise, equipment (saws, drills, electric power tools, pneumatic tools, electric power supplies, electric generators, power distribution devices such as spider boxes, work lights, temporary light bars, towers etc.), utilities, or any other applicable element associated with worksite 114 configured to affect the efficiency or overall safety of worksite 114.

In some implementations, first sensor 108 and second sensor 112 are configured to collect a plurality of environmental data, wherein environmental data is associated with a component of worksite 114 (personnel 110, equipment, utility, etc.). As described herein, environmental data may include, but is not limited to a temperature, location data (GPS data), an atmospheric humidity, an air quality measurement, an operational status, a windspeed, audio data, gas/chemical presence, or any other applicable data configured to be associated with an environment. In some implementations, the temperature data may include an ambient temperature, an outside temperature, a change in temperature over time, such as an hour or a work shift. In some implementations, the location data may include Global Positioning Satellite (GPS) data, active or passive radio frequency identification (RFID) position data, Wi-Fi location data, or mobile phone position data.

Environmental data may further include tool data such as a present location of the component, a history of the location data of the component at worksite 114, on/off time of the component, usage info about the component, a power level of the component, e.g., a present battery charge level for battery operated components, maintenance information about the component, such as a maintenance history or a maintenance schedule or data of the component, a present working condition of the component, e.g., if the is functioning properly or is currently experiencing a malfunction, such as a mechanical jam or power overload.

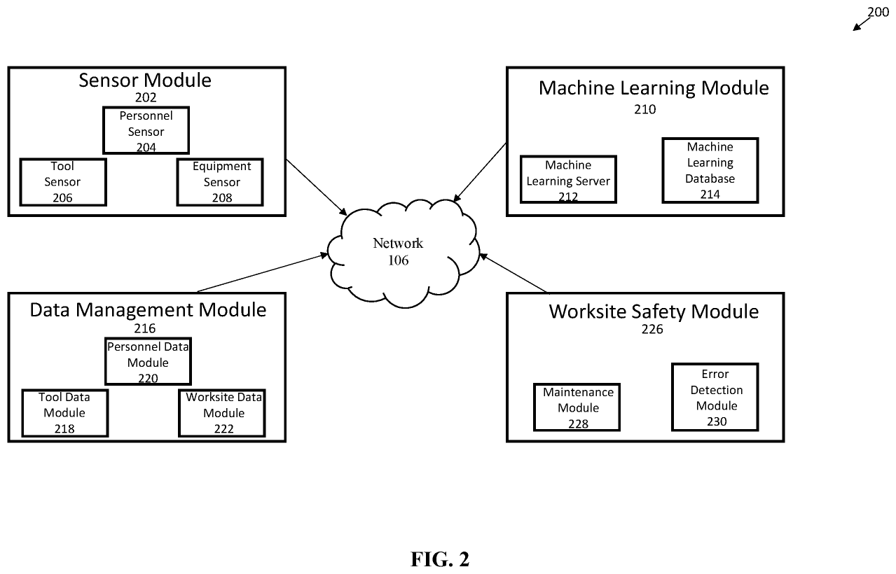

Referring now to FIG. 2, a plurality of modules 200 utilized by system 100 is depicted, according to an exemplary implementation. In one implementation, plurality of modules 200 includes a sensor module 202 including a personnel sensor 204, a tool sensor 206, and an equipment sensor 208; a machine learning module 210 including a machine learning server 212 and a machine learning database 214; a data management module 216 including a tool/equipment data module 218, a personnel data module 220, and a worksite data module 222; worksite safety module 226 including a maintenance module 228, and an error detection module 230. In some implementations, each of the aforementioned modules are designed and configured to continuously and automatically transmit applicable data to computing device 102 over network 106. In some implementations, the modules may periodically collect data and may periodically transmit data. For example, sensor module 202 may periodically sample data from one or more sensors at worksite 114. The modules may have a one-to-one ratio of measurements to transmissions, or the modules may collect data and compile the data for transmission in batches.

In some implementations, sensor module 202 is directly associated with first sensor 108 and second sensor 112 wherein first sensor 108 is associated with personnel sensor 204 and second sensor 112 is associated with tool sensor 206 and/or equipment sensor 208. In some implementations, sensor module 202 may be designed and configured to collect and transmit environmental data to computing device 102 in which the data collected from the applicable sensor depends on the source the data is being collected from. For example, personnel sensor 204 may be configured to collect personnel data. In some implementations, personnel data may include vital data, location data, movement data, safety data, etc. associated with personnel 110. In some implementations, the aforementioned data may be viewable by personnel 110 via the centralized platform provided by computing device 102. Messages/notifications relating to the aforementioned data may be communicated to personnel 110 via a graphical user interface allocated on computing device 102. In some implementations, computing device 102 may include first sensor 108, such as when computing device 102 is a wearable device. Vital data may include biometric measurements, such as body temperature, respiration rate, perspiration rate, hydration, blood pressure, pulse rate, blood oxygen saturation, etc. Vital data may be used to monitor the health of personnel 110, the safety of personnel 110, dangerous conditions in personnel 110, and may be compiled for research purposes. Location information may include time stamps information such as when personnel 110 enters the worksite, leaves the worksite, when personnel 110 uses the restroom, and other location-based events. Such location-based events may be useful for time keeping, time tracking, rest break recordation, etc. The location of personnel 110 may be useful in case of an emergency; the location of personnel 110 may be transmitted to emergency services in case of a worksite emergency. Safety data may include fall detection, body position information, such as correct posture when lifting, load-detection regarding an amount being lifted, proximity of personnel 110 to others, proximity of personnel 110 to tools or equipment, proximity of personnel 110 to a dangerous condition, detection of potential hazards, such as the presence of electrical circuits or current, or other safety related information. In some implementations, detection of a safety situation may cause computing device 102 to transmit a safety alert to a sensor or communication device worn by personnel 110, such as an evacuation signal or warning signal when a dangerous situation exists at the worksite. By way of non-limiting example, such a situation may include an elevated carbon monoxide level, an excessive heat condition, a fire, or other dangerous situation of which personnel 110 should be made aware. In other implementations, computing device 102 may transmit an alarm when personnel 110 undertakes a task that is dangerous, such as lifting too heavy of a load or working near an electrical circuit or current, or when personnel 110 undertakes a task in a dangerous manner, such as lifting a load with improper posture.

In some implementations, posture may be measured or determined using the relative position of two or more sensors worm by personnel 110. In other implementations, the relative position of sensors worn by personnel 110 may be used to determine the position of personnel 110 or the motion of personnel 110. An individual moving forward may have a different configuration of sensors compared to when that individual is standing still or holding a heavy load.

In some implementations, communication signals generated by computing device 102 may cause a message to be communicated to personnel 110 using a visual signal, audible signal, or haptics. Visual signals may be messages displayed on a screen, shown by color-coded LEDs, such as by LED tape or LED bulbs, communicated by various blink-patterns of lighting element, or other visual communication means. Audible signals may be produced by speakers in a headset, speakers in a wearable device, such as a smart-watch, or a speaker in another device possessed by personnel 110. Haptic signals may be produced by haptic motors in any of the various devices personnel 110 may have.

In some implementations, first sensor 108 and/or second sensor 112 may receive and/or transmit a distress signal. The distress signal may be automated or may be in response to a user input. User input may be used to record personnel 110 recording the completion of a task, beginning, or ending a work shift, beginning or ending a meal or rest break, etc. First sensor 108 and/or second sensor 112 may send or receive emergency alerts, distress signals, or other safety related signals. In some implementations, emergency alerts or distress signals may be passive or automated, such as upon detection of a fall, or may be active, such as when the signal is initiated by a user input.

In some implementations, environmental data collected by first sensor 108 and/or second sensor 112 may be configured to be processed by computing device 102 allowing computing device 102 to generate one or more executable instructions configured to adjust the component associated with first sensor 108 and/or second sensor 112. In some implementations, the applicable component may not be readily identifiable via computing device 102 based solely off of data collected from modules 200; thus, in some implementations, computing device 102 is configured to extract an identifier associated with the component by filtering the environmental data. The identifiers may allow computing device 102 to continuously monitor the component within worksite 114 wherein computing device 102 is configured to timestamp and apply other applicable analytical features to the environmental data based on the extracted identifier. In some implementations, computing device 102 utilizes the collected environmental data to establish an operational threshold for the applicable component along with determine an efficiency score associated with the applicable component. In some implementations, the operational threshold may represent an overall level of functioning associated with a component within worksite 114. For example, a HVAC device within worksite 114 may usually maintain an ambient temperature of 70° F. wherein computing device 102 would determine based on continuously collected environmental data from the HVAC device that the operational threshold is 70° F.; however, due to a tripped breaker the HVAC device is running a temperature greater than 70° F. in which computing device 102 detects the exceeding of the operational threshold from the collected environmental data and tracking of the identifier, and automatically notifies administrator 116 of the issue.

In some implementations, machine learning module 210 is configured to utilize machine learning server 212 to apply one or more machine learning algorithms in order to generate predictions relating to components within worksite 114, wherein the predictions are configured to be utilized by computing device 102 to generate executable instructions configured to adjust the component based on the environmental data. In one implementation, machine learning server 212 may utilize a machine learning model or a rule-based model in order to generate the predictions. For example, if the model is a machine-learned model, then one or more machine learning techniques are used to “learn” weights of different features, which weights are then utilized by machine learning server 212. Machine learning server 212 may be configured to generate a classification model generated based on training data utilizing the one or more aforementioned machine learning techniques, wherein the feature values are configured to be inserted into the classification model.

Machine learning is the study and construction of algorithms that can learn from, and make predictions based on, data. Such algorithms operate by building a model from inputs in order to make data-driven predictions or decisions. Thus, a machine learning technique may be used to generate a statistical model that is trained based on a history of attribute values associated with data utilized within system 100. Training data sets may be used to train the one or more machine learning algorithms. The training data may be sourced from the environmental data including a plurality of training instances sourced from the environmental data, each of which include a plurality of feature values and a label that indicates whether the training instance pertains to the applicable component of worksite 114. Ultimately, techniques associated with the machine learning trains one or more classification models based on the training data or environmental data. The training data is configured to be dynamically acquired over long periods of time. For example, a new machine-learned model is generated regularly, such as every hour, day, month, week, or other time period. Thus, the new machine-learned model may replace a previous machine-learned model. Newly acquired or changed training data may be used to update the model. In some implementations, machine learning database 214 may function as a repository or database for the training data entered into the one or more machine learning algorithms.

In one implementation, data management module 216 oversees all data collected by sensor module 202 allowing one or more component specific records to be generated and maintained by data management module 216 and stored in database 104. In some implementations, the one or more component specific records are identified based on the identifier associated with the component allowing analytics to be performed on the specific component. For example, personnel data module 220 may generate data and data analyses specific to the HVAC collected over various periods of time which may be stored within a component specific record housed in database 104, wherein said data may be inserted into the one or more machine learning algorithms in order for a prediction to be generated associated with the HVAC.

In one implementation, worksite safety module 226 manages cumulative data associated with worksite 114 that is continuously being collected by first sensor 108 and second sensor 112. For example, first sensor 108 may be collecting data specific to personnel 110 while second sensor 112 may be collecting data specific to equipment within worksite 114 wherein personnel 110 and the component are located within a same particular room. Computing device 102 will utilize environmental data to determine an operational threshold associated with the equipment establishing a standard for operational functioning of the equipment (normal state the component should be operating). In the situation where administrator 116 wishes to know the overall efficiency of the particular room, computing device 102 may rely on historical data sourced from tool/equipment data module 218, personnel data module 220, and worksite data module 222 in order to generate the efficiency scores and logistics schedules for both the component, personnel 110, and particular room based on the efficiency scores, operational thresholds, and environmental data. In some implementations, computing device 102 may utilize the environmental data to generate vital information summaries associated with personnel 110, create reports, flag issues such as poor hydration or improper time usage, generate safety reports based on incidents catalogued, inspections performed, and customizable safety parameters.

In some implementations, maintenance module 228 maintains the operational threshold for each applicable component within worksite 114, and error detection module 230 communicates with maintenance module 228 in order to assist computing device 102 in determining if the operational threshold has been exceeded. The operational threshold may be determined to be exceeded in a variety of ways. For example, exceeding of the operational threshold may be determined based on failure to perform preventative maintenance on the component, overheating, condition-based monitoring, or any other applicable indicator that illustrates a component not operating at the standard functionality. In some implementations, the efficiency scores may be generated by computing device 102 based on the predictions generated by machine learning server 212, wherein the efficiency score is configured to represent how effective personnel 110 and/or the applicable component associated with second sensor 112 is. First sensor 108 may continuously or periodically acquire motion data, heartrate activity, and other applicable data from personnel 110 over a period of time allowing machine learning server 212 to generate predictions relating to the amount of time personnel 110 is idle when present within worksite 114. Computing device 102 may receive the predictions and analyses over network 106. In some implementations, computing device 102 may generate an efficiency score reflecting the productivity of personnel 110 within worksite 114 based on the predictions and analyses. In another example, second sensor 112 collects environmental data from a machinery (component) within worksite 114 wherein the environmental data indicated that the machinery is emitting an abnormally high temperature. Machine learning server 212 utilizes the environmental data to predict when the machinery will overheat or when and if the machinery will require servicing. Computing device 102 utilizes the predictions generated over time and the instances of the operational threshold being exceeded to generate the efficiency score for the machinery, and subsequently provides at least a graphical user interface to administrator 116 depicting the efficiency score.

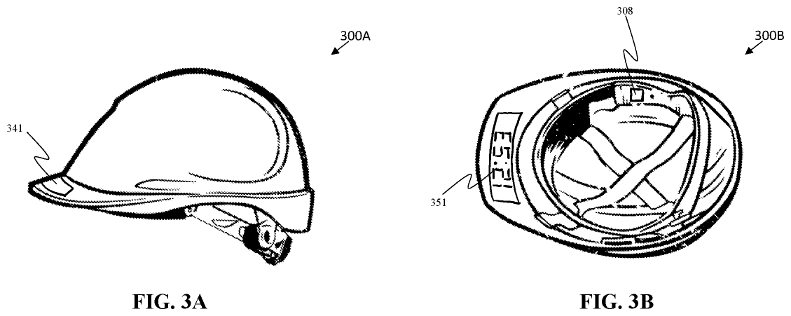

Referring now to FIG. 3A, an apparatus 301 utilized by system 100 is depicted, according to an exemplary implementation. As shown in FIG. 3, apparatus 301 is a hardhat. In other implementations, apparatus 301 may be a smart watch, chest strap, smart glove, smart boot, wearable device, or any other mechanism configured to be donned by a user. A smart device, such as a smart glove or smart boot, may include a sensor, a hardware processor, a non-transitory memory, and/or a wireless communication element. In some implementations, apparatus 301 includes a camera, headlight, light on the underside of the brim of apparatus 301, sensors to detect a change in the body position of personnel 110 wearing apparatus 301, body temperature sensors for measuring the body temperature of personnel 110 wearing apparatus 301, a mini USB charging port for charging an electrical power source of apparatus 301, a color changing LED band affixed to the upper side of the brim of apparatus 301, a microphone, and speakers. Apparatus 301 is a safety device worn by a personnel 110 present at a worksite. In some implementations, apparatus 301 may be made from a hard and preferably light-weight material, such as a thermoplastic, fiberglass, high-density polyethylene (HDPE) or advanced engineering resins, such as polyetherimide or polyether ether ketone. In some implementations, apparatus 301 may include a battery pack, a rechargeable battery pack, a photovoltaic power supply, a combination of battery and photovoltaic electric power, or other electrical power source.

In some implementations, apparatus 301 may include one or more communication elements, such as speakers, motors for providing haptic feedback, LED bulbs, an LED tape attached to an under-side of a brim of apparatus 301 situated to be visible to the worker wearing the apparatus, an LED tape attached to an upper side of the brim situated to be visible. Each communication element may be used for communication and directives. As shown in FIG. 3A, apparatus 301A includes communication element 341. Communication element 341 may be an LED tape display. The LED may be configured to provide visual information, such as using colors to communicate conditions. For example, the LED may illuminate green if conditions are all good, yellow to indicate a warning (e.g., proximity alarm, heat condition, rising CO, or smoke), red to indicate an emergent situation (high CO, fire, etc.). The visual indicator may be used to communicate messages to the worker wearing the apparatus or may communicate information about personnel 110, for example, who is looking from afar. For example, the audio/visual components of apparatus 301 may be used to send a distress or SOS signal if the worker experiences an emergency. In some implementations, computing device 102 tracks the location of apparatus 301 to ensure personnel 110 is not within an authorized/restricted area of worksite 114.

Referring now to FIG. 3B, another view of apparatus 301 shown in FIG. 3A. As shown in FIG. 3B, apparatus 301B includes a communication element 351 situated on the underside of the brim. In some implementations, display panel 351 may be an LED tape display. Display panel 351 may display information to the individual wearing apparatus 301 such as the time, environmental information, text messages from a connected smart device, management messages, error messages, safety messages, or emergency messages. Sensor 308 may be an environmental sensor, a biometric sensor, or other sensor for measuring position, location, or motion. In some implementations, apparatus 301 may include a power source (not shown) for powering sensor 308. Apparatus 301 may include a wireless communication element (not shown) for transmitting and receiving data and signals.

In other implementations, apparatus 301 may be smart watch, a chest strap, a smart glove, a smart boot, or other equipment worn by a worker at a construction site. Apparatus 301 may include to one or more sensors connected to a communication element for transmitting data gathered from the sensors. The sensors may include pressure sensors included in a boot or other footwear, a biometric sensor for gathering vital information about the individual wearing apparatus 301, an environmental sensor for gathering environmental data.

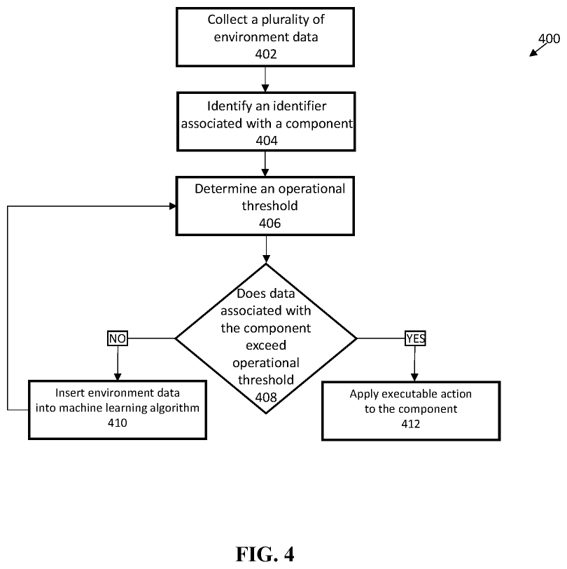

Referring now to FIG. 4, an exemplary method of managing a worksite 114 is depicted, according to an exemplary implementation. At step 401, first sensor 108 and/or second sensor 112 collect the environmental data in which environmental data may be transmitted to the plurality of modules 200 or directly to computing device 102 subject to the use of the particular environmental data. For example, computing device 102 may receive the environmental data directly in situations where the operational threshold has not been established for a particular component or when the component that the environmental data is associated with has not yet been identified by computing device 102.

At step 403, computing device 102 identifies the identifier associated with the applicable component in which computing device 102 is receiving the environmental data for. In some implementations, the identifier is a unique descriptive mechanism necessary to determine the source of the environmental data and in certain implementations the identifier may be assigned to personnel 110, a component, and/or an area within worksite 114 including personnel 110 and/or the component. In some implementations, the efficiency score generated by computing device 102 is directly associated with the identifier allowing administrator 116 to monitor the progression or regression of efficiency associated with the applicable component and/or worksite 114 overall.

At step 405, computing device 102 determines the operational threshold for the applicable component of worksite 114 based on the environmental data and the identifier. At step 407, computing device 102 determines whether the operational threshold associated with the applicable component has been exceeded based on the environmental data. For example, environmental data collected by first sensor 108 may allow computing device 102 to determine that the heartrate of personnel 110 is significantly higher than the normal heartrate of personnel 110 (the operational threshold); thus, personnel 110 being at great risk to sustain injury such as a stroke within worksite 114. Based on computing device 102 detecting the operational threshold associated with personnel 110, computing device 102 may automatically notify administrator 116 via the centralized platform that personnel 110 has exceeded the operational threshold and/or notify an applicable emergency responder or first responder. In another working example, the environmental data received from second sensor 112 may indicate that an area of worksite 114 includes poisonous gasses in the atmosphere based on an operational threshold associated with oxygen quality being exceeded in which computing device 102 automatically notifies all personnel within the area to evacuate and notifies the applicable emergency responder.

If the operational threshold is not exceeded, then step 409 occurs in which the environmental data collected by first sensor 108 and/or second sensor 112 is transmitted to plurality of modules 200 in order to be insert into the one or more machine learning algorithms via machine learning server 212, and computing device 102 may adjust and/or update the operational threshold for the applicable component based on the predictions generated by machine learning server 212. Otherwise, step 411 occurs in which computing device 102 applies an executable action to the applicable component based on computing device 102 detecting the operational threshold being exceeded by the currently collected environmental data. In some implementations, the executable action may include automatically notifying administrator 116, notifying the applicable emergency responder or first responder, and/or adjusting a functional operation of the applicable component within worksite 114. For example, upon environmental data collected by second sensor 112 indicating that the applicable component is overheating, the executable action rendered by computing device 102 may be powering down the applicable component in order to ensure overall safety of worksite 114.

In some implementations, a processor may execute worksite safety module 226 to determine if an event has occurred based on signals received from two or more sensors. Worksite safety module may track measurements from a plurality of sensors, including personnel sensor 204, tool sensor 206, and equipment sensor 208. If worksite safety module 226 detects certain changes in readings from the sensors, worksite safety module 226 may determine an occurrence of an event and send a message or alert based on that determination. Particular correlations may indicate particular events. For example, a detected rise in carbon monoxide levels received from an environmental sensor correlated with a decrease in blood oxygen saturation received from a vital sensor may indicate an unsafe scenario and may trigger an alert or warning. A rise in perspiration correlated with an increase in heartrate may indicate decreasing hydration. A sudden fall correlated with a decrease in blood pressure may indicate a loss of consciousness. These are but a few examples of correlated events that may trigger a message or waring from workplace safety module 226.

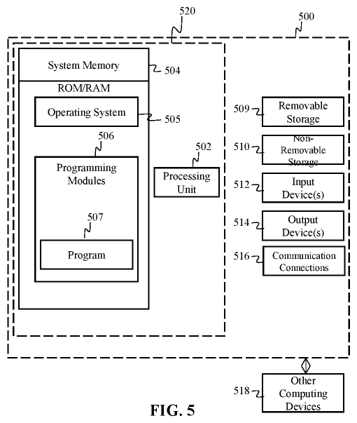

FIG. 5 is a block diagram of a system including an example computing device 500 and other computing devices. Consistent with the implementations described herein, the aforementioned actions performed by computing device 102 may be implemented in a computing device, such as the computing device 500 of FIG. 5. Any suitable combination of hardware, software, or firmware may be used to implement the computing device 500. The aforementioned system, device, and processors are examples and other systems, devices, and processors may comprise the aforementioned computing device. Furthermore, computing device 500 may comprise an operating environment for system 100 and process/method 400. Process 300, and data related to said process may operate in other environments and are not limited to computing device 500.

With reference to FIG. 5, a system consistent with an implementation of the invention may include a plurality of computing devices, such as computing device 500. In a basic configuration, computing device 500 may include at least one processing unit 502 and a system memory 504. Processing unit 502 is a hardware processor, such as a central processing unit (CPU) found in computing devices. System memory 504 is a non-transitory storage device for storing computer code for execution by processing unit 502, and for storing various data and parameters. Depending on the configuration and type of computing device, system memory 504 may comprise, but is not limited to, volatile (e.g., random access memory (RAM)), non-volatile (e.g., read-only memory (ROM)), flash memory, or any combination or memory. System memory 504 may include operating system 505, and one or more programming modules 506. Operating system 505, for example, may be suitable for controlling computing device 500's operation. In one implementation, programming modules 506 may include, for example, a program module 507 for executing the actions of computing device 102, for example. Furthermore, implementations of the invention may be practiced in conjunction with a graphics library, other operating systems, or any other application program and is not limited to any particular application or system. This basic configuration is illustrated in FIG. 5 by those components within a dashed line 520.

Computing device 500 may have additional features or functionality. For example, computing device 500 may also include additional data storage devices (removable and/or non-removable) such as, for example, magnetic disks, optical disks, or tape. Such additional storage is illustrated in FIG. 5 by a removable storage 509 and a non-removable storage 510. Computer storage media may include volatile and nonvolatile, removable and non-removable media implemented in any method or technology for storage of information, such as computer readable instructions, data structures, program modules, or other data. System memory 504, removable storage 509, and non-removable storage 510 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other non-transitory memory medium which can be used to store information, and which can be accessed by computing device 500. Any such computer storage media may be part of device 500. Computing device 500 may also have input device(s) 512 such as a keyboard, a mouse, a pen, a sound input device, a camera, a touch input device, etc. Output device(s) 514 such as a display, speakers, a printer, etc. may also be included. The aforementioned devices are only examples, and other devices may be added or substituted.

Computing device 500 may also contain a communication connection 516 that may allow device 500 to communicate with other computing devices 518, such as over a network in a distributed computing environment, for example, an intranet or the Internet. Communication connection 516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 504, including operating system 505. While executing on processing unit 502, programming modules 506 (e.g., program module 507) may perform processes including, for example, one or more of the stages of the process 400 as described above. The aforementioned processes are examples, and processing unit 502 may perform other processes. Other programming modules that may be used in accordance with implementations of the present invention may include electronic mail and contacts applications, word processing applications, spreadsheet applications, database applications, slide presentation applications, drawing or computer-aided application programs, etc.

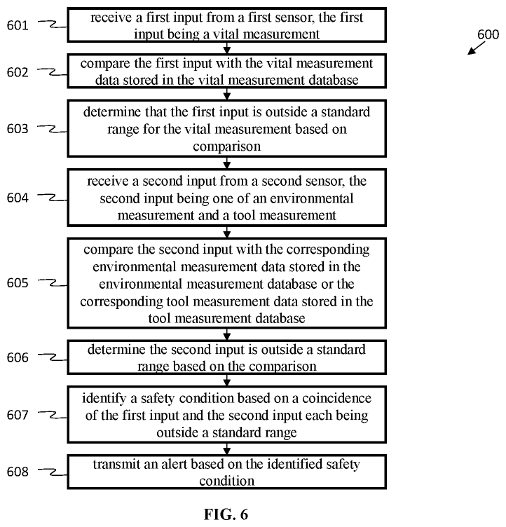

Referring now to FIG. 6, an exemplary method of managing a worksite 114 is depicted, according to an exemplary implementation. At 601, processing unit 502 receives a first input from a personnel sensor, the first input being a vital measurement. The vital measurement may reflect a current health state of personnel 110. For example, the input may include one or more vital measurements of personnel 110 in real time. In some implementations, database 104 may store a personnel profile associate with personnel 110. The personnel profile may include various data, such as baseline vital data or a history of various vital data measurements of personnel 110. Based on the data stored in the personnel profile associated with personnel 110, database 104 may include a standard range for each of the vital data associated with personnel 110.

At 602, processing unit 502 compares the first input with the vital measurement data stored in the vital measurement database. At 603, processing unit 502 determines that the first input is outside a standard range for the vital measurement based on comparison. For example, the first input may include a current blood pressure measurement of personnel 110. The current blood pressure measurement may be above or below a range of blood pressure measurements stored in the personnel profile of personnel 110. Based on the comparison of present to historical data, processing unit 502 may determine the present reading is outside of a standard range for personnel 110. Similar readings and comparisons apply to other vital data associated with personnel 110.

At 604, processing unit 502 receives a second input from a second sensor, the second input being one of an environmental measurement and a tool measurement. In some implementations, the second input is an environmental measurement, such as an ambient temperature, an ambient gas concentration, or other environmental measurement. In other implementations, the second input is a tool measurement, such as a present operational state of a tool or equipment. The present operational state may indicate the tool or equipment is functioning well or may indicate a malfunction.

At 605, processing unit 502 compares the second input with the corresponding environmental measurement data stored in the environmental measurement database or the corresponding tool measurement data stored in the tool measurement database. In some implementations, database 104 may include environmental data, such as acceptable temperature ranges, acceptable gas concentrations for various gasses, and acceptable measurements for other environmental data. In other implementations, database 104 may include various tool data, such as acceptable operational temperatures, acceptable operational time, acceptable operational speeds, acceptable operational gas concentrations (e.g., certain tools or equipment may require certain concentrations of oxygen to be safe or effective), acceptable durations between services, or other data associated with tools and equipment of worksite 114. At 606, processing unit 502 determines the second input is outside a standard range based on the comparison.

At 607, processing unit 502 identifies a safety condition based on a coincidence of the first input and the second input each being outside a standard range. In some implementations, database 104 may store correlation data between certain readings and probable conditions associated with them. For example, database 104 may show that a decrease in ambient oxygen concentration and an increase in personnel 110's respiratory rate indicates a dangerous condition. Another example may be that a change in the operational condition of a tool and a drop in blood pressure of personnel 110 may indicate an injury. These are merely examples and do not capture the full range and possible iterations of correlated data pairings indicating a safety condition exists.

At 608, processing unit 502 transmits an alert based on the identified safety condition. In some implementations, the alert may be an audio alert, a visual alert, a computer signal alerts, or an emergency call. An audio alert may play over speakers for everyone in worksite 114 to hear, or it may be broadcast wirelessly for individuals to hear. In some implementations, an alert may apply to a subsection of individuals present at worksite 110 and only those who need to be alerted will receive the alert. Visual alerts may include flashing lights at worksite 114 or visual indicator displayed individually to one or more individuals present at worksite 114. A computer signal alert may be a signal sent to a network-connected tool or equipment to shut down, cease operation, or change operational states. In some implementations, a computer signal may shut down a saw, shut down an arc-welder, deactivate a piece of equipment, or activate a fan. These are merely examples and do not capture the full range and possible iterations of actions or effects an alert could include.

From the above description, it is manifest that various techniques can be used for implementing the concepts described in the present application without departing from the scope of those concepts. Moreover, while the concepts have been described with specific reference to certain implementations, a person having ordinary skill in the art would recognize that changes can be made in form and detail without departing from the scope of those concepts. As such, the described implementations are to be considered in all respects as illustrative and not restrictive. It should also be understood that the present application is not limited to the particular implementations described above, but many rearrangements, modifications, and substitutions are possible without departing from the scope of the present disclosure.

What is claimed is:

1. A system comprising:
a plurality of sensors;
a computing device communicatively coupled to the plurality of sensors, wherein the computing device includes a non-transitory memory storing an executable code and a hardware processor executing the executable code to:
receive a plurality of environmental data associated with a worksite from one or more of the plurality of sensors;
extract at least one identifier associated with a component of the worksite from the plurality of environmental data;
identify an operational threshold based on the plurality of environmental data;
determine, based on the identifier, whether the component exceeds the operational threshold; and
transmit a notification based on the determination.

2. The system of claim 1, wherein the computing device is associated with a plurality of nodes communicatively coupled via a network.

3. The system of claim 1, wherein the plurality of environmental data comprises at least one of a temperature, an atmospheric humidity, an air quality measurement, an operational status, a windspeed, a biometric reading, a movement, a change of movement, an audio input, or a toxins reading.

4. The system of claim 1, wherein the computing device is further configured to determine an efficiency score associated with the component based on the plurality of environmental data.

5. The system of claim 1, wherein the computing device is further configured to generate a logistics schedule based on the efficiency score and the plurality of environmental data.

6. The system of claim 1, wherein the plurality of sensors comprises at least one of a camera, a scanner, an accelerometer, a gyroscope, a microphone, a thermometer, a measurement device, a transducer, a pressure switch, a capacitance switch, an electrochemical detector, a semiconductor measurer, an air quality sensor, wind speed sensor, a pressure sensor, or a photo sensor.

7. The system of claim 1, wherein the component is at least one of an individual, a safety equipment, a computing device, and a utility equipment, and wherein the component is associated with the worksite.

8. The system of claim 1, wherein the computing device is further configured to:
store training data that comprises a plurality of training instances, wherein each of the plurality of training instances includes a plurality of feature values and a label that indicates whether the training instance pertains to the component;
use one or more machine learning techniques to train a classification model based on the training data.

9. The system of claim 1, wherein the computing device is further configured to notify a first responder based on an alert indicating the identifier exceeding the operational threshold is a hazard associated with the worksite.

10. A method comprising:
receiving a plurality of environmental data associated with a worksite from one or more of a plurality of sensors;
identifying, using at least a computing device communicative coupled to the network, at least one identifier associated with a component of the worksite from the plurality of environmental data;
identifying, using the computing device, an operational threshold associated with the component based on the plurality of environmental data;
determining, using the computing device, whether the component exceeds the operational threshold based on the identifier; and
transmitting, using the computing device, a notification based on the determination.

11. The method of claim 10, wherein the plurality of sensors is associated with a plurality of nodes communicatively coupled via the network.

12. The method of claim 10, wherein the plurality of environmental data comprises at least one of a temperature, an atmospheric humidity, an air quality measurement, an operational status, a windspeed, a biometric reading, a movement, a change of movement, an audio input, or a toxins reading.

13. The method of claim 10, wherein the computing device is further configured to determine an efficiency score associated with the component based on the plurality of environmental data.

14. The method of claim 13, wherein the computing device is further configured to generate a logistics schedule based on the efficiency score and the plurality of environmental data.

15. The method of claim 10, wherein the plurality of sensors comprises at least one of a camera, a scanner, an accelerometer, a gyroscope, a microphone, a thermometer, a measurement device, a transducer, a pressure switch, a capacitance switch, an electrochemical detector, a semiconductor measurer, an air quality sensor, wind speed sensor, a pressure sensor, and a photo sensor.

16. The method of claim 10, wherein the component is at least one of an individual, a safety equipment, a computing device, or a utility equipment associated with the worksite.

17. The method of claim 10, further comprising:
storing training data that comprises a plurality of training instances in a non-transitory memory, wherein each of the plurality of training instances includes a plurality of feature values and a label that indicates whether the training instance pertains to the component; and
using one or more machine learning techniques to train a classification model based on the training data.

18. The method of claim 10, wherein the computing device is further configured to notify a first responder based on an alert indicating the identifier exceeding the operational threshold is a hazard associated with the worksite.

19. The method of claim 11, wherein determining whether the component exceeds the operational threshold based on the identifier further comprises:
applying, via the computing device, an adjustment action to the component via at least one node of the plurality of nodes.

20. A worksite safety system including a computer system comprising:
a non-transitory memory storing an executable code, a vital measurement database storing a plurality of vital measurement data, and at least one of an environmental measurement database storing a plurality of environmental measurement data and a tool measurement database storing a plurality of tool measurement data; and
a hardware processor executing the executable code to:
receive a first input from a first sensor, the first input being a vital measurement;
compare the first input with the vital measurement data stored in the vital measurement database;
determine that the first input is outside a standard range for the vital measurement based on comparison;
receive a second input from a second sensor, the second input being one of an environmental measurement and a tool measurement;
compare the second input with the corresponding environmental measurement data stored in the environmental measurement database or the corresponding tool measurement data stored in the tool measurement database;
determine the second input is outside a standard range based on the comparison;
identify a safety condition based on a coincidence of the first input and the second input each being outside a standard range; and
transmit an alert based on the identified safety condition.
